(19) United States
(12) Patent Application Publication
(10) Pub. No.: US 2012/0263713 A1
(43) Pub. Date: Oct. 18, 2012

(54) Combination therapy of an afucosylated CD20 antibody with fludarabine and/or mitoxantrone

(76) Inventors: Martin Dreyling, Planegg (DE); Daniel Alexander Heinrich, Muenchen (DE); Frank Herting, Penzberg (DE); Christian Klein, Bonstetten (CH)

(21) Appl. No.: 13/490,351

(22) Filed: Jun. 6, 2012

Related U.S. Application Data
(63) Continuation of application No. 12/855,951, filed on Aug. 13, 2010, now abandoned.

(30) Foreign Application Priority Data
Aug. 14, 2009 (EP) EP09010488.6
May 10, 2010 (EP) EP10162391.6

(51) Int. Cl. A61K 39/395; A61P 35/00

(52) U.S. Cl. 424/133.1

(57) ABSTRACT

The present invention is directed to the combination therapy of an afucosylated anti-CD20 antibody with fludarabine and/or mitoxantrone for the treatment of cancer, especially to the combination therapy of CD20 expressing cancers with an afucosylated humanized B-Ly1 antibody with fludarabine and/or mitoxantrone.

### PRIORITY TO RELATED APPLICATION(S)

This application is a continuation of U.S. patent application Ser. No. 12/855,951, filed Aug. 13, 2010, which claims the benefit of European Patent Application No. 09010488.6, filed Aug. 14, 2009, and European Patent Application No. 10162391.6, filed May 10, 2010, all of which are hereby incorporated by reference in their entirety.

### BACKGROUND OF THE INVENTION

The present invention is directed to the combination therapy of an afucosylated CD20 antibody with fludarabine and/or mitoxantrone for the treatment of cancer.

### Afucosylated Antibodies

Cell-mediated effector functions of monoclonal antibodies can be enhanced by engineering their oligosaccharide component as described in Umaña, P., et al., Nature Biotechnol. 17 (1999) 176-180 and U.S. Pat. No. 6,602,684. IgG1 type antibodies, the most commonly used antibodies in cancer immunotherapy, are glycoproteins that have a conserved N-linked glycosylation site at Asn297 in each CH2 domain. The two complex biantennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC) (Lifely, M. R., et al., Glycobiology 5 (1995) 813-822; Jefferis, R., et al., Immunol. Rev. 163 (1998) 59-76; Wright, A, and Morrison, S. L., Trends Biotechnol. 15 (1997) 26-32). Umaña, P., et al. Nature Biotechnol. 17 (1999) 176-180 and WO1999/54342 showed that overexpression in Chinese hamster ovary (CHO) cells of β(1,4)-N-acetylglucosaminyltransferase III (“GnTIII”), a glycosyltransferase catalyzing the formation of bisected oligosaccharides, significantly increases the in vitro ADCC activity of antibodies. Alterations in the composition of the N297 carbohydrate or its elimination affect also binding to Fc binding to FcγR and C1q (Umaña, P., et al., Nature Biotechnol. 17 (1999) 176-180; Davies, J., et al., Biotechnol. Bioeng. 74 (2001) 288-294; Mimura, Y., et al., J. Biol. Chem. 276 (2001) 45539-45547; Radaev, S., et al., J. Biol. Chem. 276 (2001) 16478-16483; Shields, R. L., et al., J. Biol. Chem. 276 (2001) 6591-6604; Shields, R. L., et al., J. Biol. Chem. 277 (2002) 26733-26740; Simmons, L. C., et al., J. Immunol. Methods 263 (2002) 133-147).

Studies discussing the activities of afucosylated and fucosylated antibodies, including anti-CD20 antibodies, have been reported (e.g., Iida, S., et al., Clin. Cancer Res. 12 (2006) 2879-2887; Natsume, A., et al., J. Immunol. Methods 306 (2005) 93-103; Satoh, M., et al., Expert Opin. Biol. Ther. 6 (2006) 1161-1173; Kanda, Y., et al., Biotechnol. Bioeng. 94 (2006) 680-688; Davies, J., et al., Biotechnol. Bioeng. 74 (2001) 288-294.

### CD20 and Anti CD20 Antibodies

The CD20 molecule (also called human B-lymphocyte-restricted differentiation antigen or Bp35) is a hydrophobic transmembrane protein located on pre-B and mature B lymphocytes that has been described extensively (Valentine, M. A., et al., J. Biol. Chem. 264 (1989) 11282-11287; and Einfeld, D. A., et al., EMBO J. 7 (1988) 711-717; Tedder, T. F., et al., Proc. Natl. Acad. Sci. U.S.A. 85 (1988) 208-212; Stamenkovic, I., et al., J. Exp. Med. 167 (1988) 1975-1980; Tedder, T. F., et al., J. Immunol. 142 (1989) 2560-2568). CD20 is expressed on greater than 90% of B cell non-Hodgkin's lymphomas (NHL) (Anderson, K. C., et al., Blood 63 (1984) 1424-1433) but is not found on hematopoietic stem cells, pro-B cells, normal plasma cells, or other normal tissues (Tedder, T. F., et al., J, Immunol. 135 (1985) 973-979).

There exist two different types of anti-CD20 antibodies differing significantly in their mode of CD20 binding and biological activities (Cragg, M. S., et al., Blood 103 (2004) 2738-2743; and Cragg, M. S., et al., Blood 101 (2003) 1045-1051). Type I antibodies, as, e.g., rituximab (a non-afucosylated antibody with an amount of fucose of 85% or higher), are potent in complement mediated cytotoxicity.

Type II antibodies, as e.g. Tositumomab (B1), 11B8, AT80 or humanized B-Ly1 antibodies, effectively initiate target cell death via caspase-independent apoptosis with concomitant phosphatidylserine exposure.

The sharing common features of type I and type II anti-CD20 antibodies are summarized in Table 1.

TABLE 1
Properties of type I and type II anti-CD20 antibodies

| type I anti-CD20 antibodies | type II anti-CD20 antibodies |
|---|---|
| type I CD20 epitope | type II CD20 epitope |
| Localize CD20 to lipid rafts | Do not localize CD20 to lipid rafts |
| Increased CDC (if IgG1 isotype) | Decreased CDC (if IgG1 isotype) |
| ADCC activity (if IgG1 isotype) | ADCC activity (if IgG1 isotype) |
| Full binding capacity | Reduced binding capacity |
| Homotypic aggregation | Stronger homotypic aggregation |
| Apoptosis induction upon cross- | Strong cell death induction without |
| linking | cross-linking |

### Fludarabine or Mitoxantrone

Fludarabine is [(2R,3R,4S,5R)-5-(6-amino-2-fluoro-purin-9-yl)-3,4-dihydroxy-oxolan-2-yl]methoxyphosphonic acid. It is DNA precursors/antimetabolites and functions as halogenated ribonucleotide reductase inhibitor. Fludarabine or fludarabine phosphate (Fludara) is a chemotherapy drug used in the treatment of hematological malignancies (Rai, K. R. et al., N. Engl. J. Med. 343 (2000) 1750-1757).

Mitoxantrone is 1,4-dihydroxy-5,8-bis[2-(2-hydroxyethylamino)ethylamino]-anthracene-9,10-dione. It is an Anthracenedione (not an anthracycline) agent. It is used in the treatment of certain types of cancer, mostly metastatic breast cancer, acute myeloid leukemia, and non-Hodgkin's lymphoma and multiple sclerosis (MS)a. Mitoxantrone is a type II topoisomerase inhibitor; it disrupts DNA synthesis and DNA repair in both healthy cells and cancer cells.

There have been reports of preclinical and/or clinical studies using the combination of fludarabine and/or mitoxantrone with rituximab (Tempescul, A., et al., (2009) Ann. Hematol. 88: 85-88; Tobinai, K., et al., Cancer Sci. 100 (2009) 1951-1956).

### SUMMARY OF THE INVENTION

Surprisingly we have now found out that the combination of fludarabine and/or mitoxantrone (especially the in vivo combination of fludarabine) with an afucosylated anti-CD20 antibody showed synergistic (e.g. more than additive in some cases) antiproliferative effects compared to the combination with non-afucosylated CD20 antibody rituximab.

Therefore the invention comprises the use of an afucosylated anti-CD20 antibody with an amount of fucose of 60% or less, for the manufacture of a medicament for the treatment of cancer in combination with fludarabine and/or mitoxantrone.

One aspect of the invention is a method of treatment of patient suffering from cancer by administering an afucosylated anti-CD20 antibody with an amount of fucose of 60% or less in combination with fludarabine and/or mitoxantrone, to a patient in the need of such treatment.

Another aspect of the invention is an afucosylated anti-CD20 antibody with an amount of fucose of 60% or less, for the treatment of cancer in combination with fludarabine and/or mitoxantrone.

In one embodiment, the amount of fucose is between 40% and 60% of the total amount of oligosaccharides (sugars) at Asn297.

In another embodiment, the amount of fucose is 0% of the total amount of oligosaccharides (sugars) at Asn297.

In one embodiment, the afucosylated anti-CD20 antibody is an IgG1 antibody.

In another embodiment, said afucosylated anti-CD20 antibody is humanized B-Ly1 antibody, and said cancer is a CD20 expressing cancer, which in one embodiment is a B-Cell Non-Hodgkin's lymphoma (NHL).

In one embodiment, the afucosylated anti-CD20 antibody binds CD20 with an KD of 10⁻⁹ M to 10⁻¹³ mol/l.

In one embodiment the treatment of cancer is in combination with fludarabine.

In one embodiment the treatment is characterized in that the humanized B-Ly1 antibody is administered in a dosage of 800 to 1600 mg on day 1 of up to six or seven 3- to 4-week-dosage-cycles, and fludarabine is administered in a dosage of 20 mg/m² to 30 mg/m² on day 1, 2 and 3 of up to six or seven 4-week-dosage-cycles.

In one embodiment the treatment is in combination with fludarabine and cyclophosphamide.

In one embodiment the treatment of cancer is characterized in that the humanized B-Ly1 antibody is administered in a dosage of 800 to 1600 mg on day 1 of up to six or seven 3- to 4-week-dosage-cycles, fludarabine is administered in a dosage of 20 mg/m² to 30 mg/m² on day 1, 2 and 3 of up to six or seven 4-week-dosage-cycles, and cyclophosphamide is administered in a dosage of 200 mg/m² to 300 mg/m² on day 1, 2 and 3 of up to six or seven 4-week-dosage-cycles.

In one embodiment the treatment of cancer is in combination with mitoxantrone.

In one embodiment the treatment of cancer is characterized in that one or more additional other cytotoxic, chemotherapeutic or anti-cancer agents, or compounds or ionizing radiation that enhance the effects of such agents are administered.

One embodiment of the invention is a composition comprising an anti-CD20 afucosylated antibody with an amount of fucose of 60% or less, and fludarabine and/or mitoxantrone (preferably fludarabine) for the treatment of cancer.

### DETAILED DESCRIPTION OF THE INVENTION

The invention comprises the use of an afucosylated anti-CD20 antibody (of IgG1 or IgG3 isotype) with an amount of fucose of 60% or less of the total amount of oligosaccharides (sugars) at Asn297, for the manufacture of a medicament for the treatment of cancer in combination with fludarabine and/or mitoxantrone.

In one embodiment, the amount of fucose is between 40% and 60% of the total amount of oligosaccharides (sugars) at Asn297.

The term “antibody” encompasses the various forms of antibodies including but not being limited to whole antibodies, human antibodies, humanized antibodies and genetically engineered antibodies like monoclonal antibodies, chimeric antibodies or recombinant antibodies as well as fragments of such antibodies as long as the characteristic properties according to the invention are retained. The terms “monoclonal antibody” or “monoclonal antibody composition” as used herein refer to a preparation of antibody molecules of a single amino acid composition. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic non-human animal, e.g. a transgenic mouse, having a genome comprising a human heavy chain transgene and a light human chain transgene fused to an immortalized cell.

The term “chimeric antibody” refers to a monoclonal antibody comprising a variable region, i.e., binding region, from one source or species and at least a portion of a constant region derived from a different source or species, usually prepared by recombinant DNA techniques. Chimeric antibodies comprising a murine variable region and a human constant region are especially preferred. Such murine/human chimeric antibodies are the product of expressed immunoglobulin genes comprising DNA segments encoding murine immunoglobulin variable regions and DNA segments encoding human immunoglobulin constant regions. Other forms of “chimeric antibodies” encompassed by the present invention are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art. See, e.g., Morrison, S. L., et al., Proc. Natl. Acad. Sci. USA 81 (1984) 6851-6855; U.S. Pat. No. 5,202,238 and U.S. Pat. No. 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See, e.g., Riechmann, L., et al., Nature 332 (1988) 323-327; and Neuberger, M. S., et al., Nature 314 (1985) 268-270.

The term “human antibody”, as used herein, is intended to include antibodies having variable and constant regions derived from human germline immunoglobulin sequences. Human antibodies are well-known in the state of the art (van Dijk, M. A., and van de Winkel, Curr. Opin. Chem. Biol. 5 (2001) 368-374). Based on such technology, human antibodies against a great variety of targets can be produced. Examples of human antibodies are for example described in Kellermann, S. A., et al., Curr. Opin. Biotechnol. 13 (2002) 593-597.

The term “recombinant human antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant human antibodies have variable and constant regions derived from human germline immunoglobulin sequences in a rearranged form. The recombinant human antibodies according to the invention have been subjected to in vivo somatic hypermutation. Thus, the amino acid sequences of the VH and VL regions of the recombinant antibodies are sequences that, while derived from and related to human germline VH and VL sequences, may not naturally exist within the human antibody germline repertoire in vivo.

As used herein, the term “binding” or “specifically binding” refers to the binding of the antibody to an epitope of the tumor antigen in an in vitro assay, preferably in an plasmon resonance assay (BIAcore, GE-Healthcare Uppsala, Sweden) with purified wild-type antigen. The affinity of the binding is defined by the terms ka (rate constant for the association of the antibody from the antibody/antigen complex), k_{D} (dissociation constant), and K_{D} (k_{D}/ka). Binding or specifically binding means a binding affinity (K_{D}) of 10⁻⁸ mol/l or less, preferably 10⁻⁹ M to 10⁻¹³ mol/l. Thus, an afucosylated antibody according to the invention is specifically binding to the tumor antigen with a binding affinity (K_{D}) of 10⁻⁸ mol/l or less, preferably 10⁻⁹ M to 10⁻¹³ mol/l.

The term “nucleic acid molecule”, as used herein, is intended to include DNA molecules and RNA molecules. A nucleic acid molecule may be single-stranded or double-stranded, but preferably is double-stranded DNA.

The “constant domains” are not involved directly in binding the antibody to an antigen but are involved in the effector functions (ADCC, complement binding, and CDC).

The “variable region” (variable region of a light chain (VL), variable region of a heavy chain (VH)) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a b-sheet conformation and the CDRs may form loops connecting the b-sheet structure. The CDRs in each chain are held in their three-dimensional structure by the framework regions and form together with the CDRs from the other chain the antigen binding site.

The terms “hypervariable region” or “antigen-binding portion of an antibody” when used herein refer to the amino acid residues of an antibody which are responsible for antigen-binding. The hypervariable region comprises amino acid residues from the “complementarity determining regions” or “CDRs”. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. Especially, CDR3 of the heavy chain is the region which contributes most to antigen binding. CDR and FR regions are determined according to the standard definition of Kabat et al., Sequences of Proteins of Immunological Interest, 5th ed., Public Health Service, National Institutes of Health, Bethesda, Md. (1991)) and/or those residues from a “hypervariable loop”.

Fludarabine is [(2R,3R,4S,5R)-5-(6-amino-2-fluoro-purin-9-yl)-3,4-dihydroxy-oxolan-2-yl]methoxyphosphonic acid. It is DNA precursors/antimetabolites and functions as halogenated ribonucleotide reductase inhibitor. Fludarabine or fludarabine phosphate (Fludara) is a chemotherapy drug used in the treatment of hematological malignancies (Rai, K. R., et al., N. Engl. J. Med. 343 (2000) 1750-1757). Fludarabine is used in various combinations with cyclophosphamide, mitoxantrone, dexamethasone and rituximab in the treatment of indolent non-Hodgkins lymphomas. As part of the FLAG regimen, fludarabine is used together with cytarabine and granulocyte colony-stimulating factor in the treatment of acute myeloid leukaemia. Because of its immunosuppressive effects, fludarabine is also used in some conditioning regimens prior to non myeloablative allogeneic stem cell transplant.

Mitoxantrone is 1,4-dihydroxy-5,8-bis[2-(2-hydroxyethylamino)ethylamino]-anthracene-9,10-dione. It is an Anthracenedione agent. It is used in the treatment of certain types of cancer, mostly metastatic breast cancer, acute myeloid leukemia, and non-Hodgkin's lymphoma. The combination of mitoxantrone and prednisone is approved as a second-line treatment for metastatic hormone-refractory prostate cancer. This combination has been the first line of treatment, until recently, when combination of docetaxel and prednisone has been shown to improve survival and disease-free period. Mitoxantrone is a type II topoisomerase inhibitor; it disrupts DNA synthesis and DNA repair in both healthy cells and cancer cells. Mitoxantrone is also used to treat multiple sclerosis (MS), most notably the subset known as secondary progressive MS. Mitoxantrone will not cure multiple sclerosis, but is effective in slowing the progression of secondary progressive MS and extending the time between relapses in relapsing-remitting MS and progressive relapsing MS.

The term “afucosylated antibody” refers to an antibody of IgG1 or IgG3 isotype (preferably of IgG1 isotype) with an altered pattern of glycosylation in the Fc region at Asn297 having a reduced level of fucose residues. Glycosylation of human IgG1 or IgG3 occurs at Asn297 as core fucosylated bianntennary complex oligosaccharide glycosylation terminated with up to 2 Gal residues. These structures are designated as G0, G1 (α1,6 or α1,3) or G2 glycan residues, depending from the amount of terminal Gal residues (Raju, T. S., BioProcess Int. 1 (2003) 44-53). CHO type glycosylation of antibody Fc parts is e.g. described by Routier, F. H., Glycoconjugate J. 14 (1997) 201-207. Antibodies which are recombinantly expressed in non glycomodified CHO host cells usually are fucosylated at Asn297 in an amount of at least 85%. It should be understood that the term an afucosylated antibody as used herein includes an antibody having no fucose in its glycosylation pattern. It is commonly known that typical glycosylated residue position in an antibody is the asparagine at position 297 according to the EU numbering system (“Asn297”).

The “EU numbering system” or “EU index” is generally used when referring to a residue in an immunoglobulin heavy chain constant region (e.g., the EU index reported in Kabat et al., Sequences of Proteins of Immunological Interest, 5th Ed. Public Health Service, National Institutes of Health, Bethesda, Md. (1991) expressly incorporated herein by reference).

Thus an afucosylated antibody according to the invention means an antibody of IgG1 or IgG3 isotype wherein the amount of fucose is 60% or less of the total amount of oligosaccharides (sugars) at Asn297 (which means that at least 40% or more of the oligosaccharides of the Fc region at Asn297 are afucosylated). In one embodiment the amount of fucose is between 40% and 60% of the oligosaccharides of the Fc region at Asn297. In another embodiment the amount of fucose is 50% or less, and in still another embodiment the amount of fucose is 30% or less of the oligosaccharides of the Fc region at Asn297. In an alternative embodiment, the amount of fucose is 0% of the oligosaccharides of the Fc region at Asn297. According to the invention “amount of fucose” means the amount of said oligosaccharide (fucose) within the oligosaccharide (sugar) chain at Asn297, related to the sum of all oligosaccharides (sugars) attached to Asn 297 (e.g. complex, hybrid and high mannose structures) measured by MALDI-TOF mass spectrometry and calculated as average value (a detailed procedure to determine the amount of fucose is described e.g. in WO2008/077546). Furthermore in one embodiment, the oligosaccharides of the Fc region are bisected. The afucosylated antibody according to the invention can be expressed in a glycomodified host cell engineered to express at least one nucleic acid encoding a polypeptide having GnTIII activity in an amount sufficient to partially fucosylate the oligosaccharides in the Fc region. In one embodiment, the polypeptide having GnTIII activity is a fusion polypeptide. Alternatively α1,6-fucosyltransferase activity of the host cell can be decreased or eliminated according to U.S. Pat. No. 6,946,292 to generate glycomodified host cells. The amount of antibody fucosylation can be predetermined e.g. either by fermentation conditions (e.g. fermentation time) or by combination of at least two antibodies with different fucosylation amount. Such afucosylated antibodies and respective glycoengineering methods are described in WO2005/044859, WO2004/065540, WO2007/031875, Umana, P., et al., Nature Biotechnol. 17 (1999) 176-180), WO1999/54342, WO2005/018572, WO2006/116260, WO2006/114700, WO2005/011735, WO2005/027966, WO97/028267, US2006/0134709, US2005/0054048, US2005/0152894, WO2003/035835, WO2000/061739. These glycoengineered antibodies have an increased ADCC. Other glycoengineering methods yielding afucosylated antibodies according to the invention are described e.g. in Niwa, R., et al., J. Immunol. Methods 306 (2005) 151-160; Shinkawa, T., et al., J. Biol. Chem. 278 (2003) 3466-3473; WO03/055993 or US2005/0249722.

Thus one aspect of the invention is the use of an afucosylated anti-CD20 antibody of IgG1 or IgG3 isotype (preferably of IgG1 isotype) specifically binding to a CD20 with an amount of fucose of 60% or less of the total amount of oligosaccharides (sugars) at Asn297, for the manufacture of a medicament for the treatment of cancer in combination with fludarabine and/or mitoxantrone. In one embodiment, the amount of fucose is between 40% and 60% of the total amount of oligosaccharides (sugars) at Asn297.

CD20 (also known as B-lymphocyte antigen CD20, B-lymphocyte surface antigen B1, Leu-16, Bp35, BM5, and LF5; the sequence is characterized by the SwissProt database entry P11836) is a hydrophobic transmembrane protein with a molecular weight of approximately 35 kD located on pre-B and maturem B lymphocytes (Valentine, M. A., et al., J. Biol. Chem. 264 (1989) 11282-11287; Tedder, T. F., et al., Proc. Natl. Acad. Sci. U.S.A. 85 (1988) 208-12; Stamenkovic, I., et al., J. Exp. Med. 167 (1988) 1975-1980; Einfeld, D. A., et al., EMBO J. 7 (1988) 711-717; Tedder, T. F., et al., J. Immunol. 142 (1989) 2560-2568). The corresponding human gene is Membrane-spanning 4-domains, subfamily A, member 1, also known as MS4A1. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes the B-lymphocyte surface molecule which plays a role in the development and differentiation of B-cells into plasma cells. This family member is localized to 11q12, among a cluster of family members. Alternative splicing of this gene results in two transcript variants which encode the same protein.

The terms “CD20” and “CD20 antigen” are used interchangeably herein, and include any variants, isoforms and species homologs of human CD20 which are naturally expressed by cells or are expressed on cells transfected with the CD20 gene. Binding of an antibody of the invention to the CD20 antigen mediate the killing of cells expressing CD20 (e.g., a tumor cell) by inactivating CD20. The killing of the cells expressing CD20 may occur by one or more of the following mechanisms: Cell death/apoptosis induction, ADCC and CDC.

Synonyms of CD20, as recognized in the art, include B-lymphocyte antigen CD20, B-lymphocyte surface antigen B1, Leu-16, Bp35, BM5, and LF5.

The term “anti-CD20 antibody” according to the invention is an antibody that binds specifically to CD20 antigen. Depending on binding properties and biological activities of anti-CD20 antibodies to the CD20 antigen, two types of anti-CD20 antibodies (type I and type II anti-CD20 antibodies) can be distinguished according to Cragg, M. S., et al., Blood 103 (2004) 2738-2743; and Cragg, M. S., et al., Blood 101 (2003) 1045-1051, see Table 2.

TABLE 2
Properties of type I and type II anti-CD20 antibodies

| type I anti-CD20 antibodies | type II anti-CD20 antibodies |
|---|---|
| type I CD20 epitope | type II CD20 epitope |
| Localize CD20 to lipid rafts | Do not localize CD20 to lipid rafts |
| Increased CDC (if IgG1 isotype) | Decreased CDC (if IgG1 isotype) |
| ADCC activity (if IgG1 isotype) | ADCC activity (if IgG1 isotype) |
| Full binding capacity | Reduced binding capacity |
| Homotypic aggregation | Stronger homotypic aggregation |
| Apoptosis induction upon cross- | Strong cell death induction without |
| linking | cross-linking |

Examples of type II anti-CD20 antibodies include e.g. humanized B-Ly1 antibody IgG1 (a chimeric humanized IgG1 antibody as disclosed in WO2005/044859), 11B8 IgG1 (as disclosed in WO2004/035607), and AT80 IgG1. Typically type II anti-CD20 antibodies of the IgG1 isotype show characteristic CDC properties. Type II anti-CD20 antibodies have a decreased CDC (if IgG1 isotype) compared to type I antibodies of the IgG1 isotype.

Examples of type I anti-CD20 antibodies include e.g. rituximab, HI47 IgG3 (ECACC, hybridoma), 2C6 IgG1 (as disclosed in WO2005/103081), 2F2 IgG1 (as disclosed and WO2004/035607 and WO2005/103081) and 2H7 IgG1 (as disclosed in WO2004/056312).

The afucosylated anti-CD20 antibodies according to the invention is in one embodiment a type II anti-CD20 antibody, in another embodiment an afucosylated humanized B-Ly1 antibody.

The afucosylated anti-CD20 antibodies according to the invention have an increased antibody dependent cellular cytotoxicity (ADCC) unlike anti-CD20 antibodies having no reduced fucose.

By “afucosylated anti-CD20 antibody with increased antibody dependent cellular cytotoxicity (ADCC)” is meant an afucosylated anti-CD20 antibody, as that term is defined herein, having increased ADCC as determined by any suitable method known to those of ordinary skill in the art. One accepted in vitro ADCC assay is as follows:

1) the assay uses target cells that are known to express the target antigen recognized by the antigen-binding region of the antibody;
2) the assay uses human peripheral blood mononuclear cells (PBMCs), isolated from blood of a randomly chosen healthy donor, as effector cells;
3) the assay is carried out according to following protocol:
i) the PBMCs are isolated using standard density centrifugation procedures and are suspended at 5×10⁶ cells/ml in RPMI cell culture medium;
ii) the target cells are grown by standard tissue culture methods, harvested from the exponential growth phase with a viability higher than 90%, washed in RPMI cell culture medium, labeled with 100 micro-Curies of ⁵¹Cr, washed twice with cell culture medium, and resuspended in cell culture medium at a density of 10⁵ cells/ml;
iii) 100 microliters of the final target cell suspension above are transferred to each well of a 96-well microtiter plate;
iv) the antibody is serially-diluted from 4000 ng/ml to 0.04 ng/ml in cell culture medium and 50 microliters of the resulting antibody solutions are added to the target cells in the 96-well microtiter plate, testing in triplicate various antibody concentrations covering the whole concentration range above;
v) for the maximum release (MR) controls, 3 additional wells in the plate containing the labeled target cells, receive 50 microliters of a 2% (VN) aqueous solution of non-ionic detergent (Nonidet, Sigma, St. Louis), instead of the antibody solution (point iv above);
vi) for the spontaneous release (SR) controls, 3 additional wells in the plate containing the labeled target cells, receive 50 microliters of RPMI cell culture medium instead of the antibody solution (point iv above);
vii) the 96-well microtiter plate is then centrifuged at 50×g for 1 minute and incubated for 1 hour at 4° C.;
viii) 50 microliters of the PBMC suspension (point i above) are added to each well to yield an effector:target cell ratio of 25:1 and the plates are placed in an incubator under 5% CO₂ atmosphere at 37 C for 4 hours;
ix) the cell-free supernatant from each well is harvested and the experimentally released radioactivity (ER) is quantified using a gamma counter;
x) the percentage of specific lysis is calculated for each antibody concentration according to the formula (ER-MR)/(MR-SR)×100, where ER is the average radioactivity quantified (see point ix above) for that antibody concentration, MR is the average radioactivity quantified (see point ix above) for the MR controls (see point V above), and SR is the average radioactivity quantified (see point ix above) for the SR controls (see point vi above);
4) “increased ADCC” is defined as either an increase in the maximum percentage of specific lysis observed within the antibody concentration range tested above, and/or a reduction in the concentration of antibody required to achieve one half of the maximum percentage of specific lysis observed within the antibody concentration range tested above. The increase in ADCC is relative to the ADCC, measured with the above assay, mediated by the same antibody, produced by the same type of host cells, using the same standard production, purification, formulation and storage methods, which are known to those skilled in the art, but that has not been produced by host cells engineered to overexpress GnTIII.

Said “increased ADCC” can be obtained by glycoengineering of said antibodies, that means enhance said natural, cell-mediated effector functions of monoclonal antibodies by engineering their oligosaccharide component as described in Umana, P., et al., Nature Biotechnol. 17 (1999) 176-180 and U.S. Pat. No. 6,602,684.

The term “complement-dependent cytotoxicity (CDC)” refers to lysis of human tumor target cells by the antibody according to the invention in the presence of complement. CDC is measured preferably by the treatment of a preparation of CD20 expressing cells with an anti-CD20 antibody according to the invention in the presence of complement. CDC is found if the antibody induces at a concentration of 100 nM the lysis (cell death) of 20% or more of the tumor cells after 4 hours. The assay is performed preferably with ⁵¹Cr or Eu labeled tumor cells and measurement of released ⁵¹Cr or Eu. Controls include the incubation of the tumor target cells with complement but without the antibody.

The “rituximab” antibody (reference antibody; example of a type I anti-CD20 antibody) is a genetically engineered chimeric human gamma 1 murine constant domain containing monoclonal antibody directed against the human CD20 antigen. This chimeric antibody contains human gamma 1 constant domains and is identified by the name “C2B8” in U.S. Pat. No. 5,736,137 (Andersen, et. al.) issued on Apr. 17, 1998, assigned to IDEC Pharmaceuticals Corporation. Rituximab is approved for the treatment of patients with relapsed or refracting low-grade or follicular, CD20 positive, B cell non-Hodgkin's lymphoma. In vitro mechanism of action studies have shown that rituximab exhibits human complement-dependent cytotoxicity (CDC) (Reff, M. E., et. al., Blood 83(2) (1994) 435-445). Additionally, it exhibits significant activity in assays that measure antibody-dependent cellular cytotoxicity (ADCC). Rituximab is not afucosylated.

| Antibody | Amount of fucose |
|---|---|
| Rituximab (non- | >85% |
| afucosylated) | |
| Wild type afucosylated | >85% |
| glyco-engineered | |
| humanized B-Ly1 (B- | |
| HH6-B-KV1) (non- | |
| afucosylated) | |
| afucosylated glyco- | 45-50% |
| engineered humanized B- | |
| Ly1 (B-HH6-B-KV1 GE) | |

The term “humanized B-Ly1 antibody” refers to humanized B-Ly1 antibody as disclosed in WO2005/044859 and WO2007/031875, which were obtained from the murine monoclonal anti-CD20 antibody B-Ly1 (variable region of the murine heavy chain (VH): SEQ ID NO: 1; variable region of the murine light chain (VL): SEQ ID NO: 2—see Poppema, S, and Visser, L., Biotest Bulletin 3 (1987) 131-139) by chimerization with a human constant domain from IgG1 and following humanization (see WO2005/044859 and WO2007/031875). These “humanized B-Ly1 antibodies” are disclosed in detail in WO2005/044859 and WO2007/031875.

In one embodiment, the “humanized B-Ly1 antibody” has variable region of the heavy chain (VH) selected from group of SEQ ID NO: 3 to SEQ ID NO: 20 (B-HH2 to B-HH9 and B-HL8 to B-HL17 of WO2005/044859 and WO2007/031875).

In one specific embodiment, such variable domain is selected from the group consisting of Seq. ID No. 3, 4, 7, 9, 11, 13 and 15 (B-HH2, B-HH3, B-HH6, B-HH8, B-HL8, B-HL11 and B-HL13 of WO2005/044859 and WO2007/031875). Preferably the “humanized B-Ly1 antibody” has variable region of the light chain (VL) of SEQ ID NO: 20 (B-KV1 of WO2005/044859 and WO2007/031875). In one specific embodiment, the “humanized B-Ly1 antibody” has a variable region of the heavy chain (VH) of SEQ ID NO: 7 (B-HH6 of WO2005/044859 and WO2007/031875) and a variable region of the light chain (VL) of SEQ ID NO: 20 (B-KV1 of WO2005/044859 and WO2007/031875). Furthermore in one embodiment, the humanized B-Ly1 antibody is an IgG1 antibody. According to the invention such afucosylated humanized B-Ly1 antibodies are glycoengineered (GE) in the Fc region according to the procedures described in WO2005/044859, WO2004/065540, WO2007/031875, Umana, P., et al., Nature Biotechnol. 17 (1999) 176-180 and WO1999/54342. In one embodiment, the afucosylated glyco-engineered humanized B-Ly1 is B-HH6-B-KV1 GE. Such glycoengineered humanized B-Ly1 antibodies have an altered pattern of glycosylation in the Fc region, preferably having a reduced level of fucose residues. In one embodiment, the amount of fucose is 60% or less of the total amount of oligosaccharides at Asn297 (in one embodiment the amount of fucose is between 40% and 60%, in another embodiment the amount of fucose is 50% or less, and in still another embodiment the amount of fucose is 30% or less). In another embodiment, the oligosaccharides of the Fc region are bisected. These glycoengineered humanized B-Ly1 antibodies have an increased ADCC.

The oligosaccharide component can significantly affect properties relevant to the efficacy of a therapeutic glycoprotein, including physical stability, resistance to protease attack, interactions with the immune system, pharmacokinetics, and specific biological activity. Such properties may depend not only on the presence or absence, but also on the specific structures, of oligosaccharides. Some generalizations between oligosaccharide structure and glycoprotein function can be made. For example, certain oligosaccharide structures mediate rapid clearance of the glycoprotein from the bloodstream through interactions with specific carbohydrate binding proteins, while others can be bound by antibodies and trigger undesired immune reactions (Jenkins, N., et al., Nature Biotechnol. 14 (1996) 975-981).

Mammalian cells are the excellent hosts for production of therapeutic glycoproteins, due to their capability to glycosylate proteins in the most compatible form for human application (Cumming, D. A., et al., Glycobiology 1 (1991) 115-130; Jenkins, N., et al., Nature Biotechnol. 14 (1996) 975-981). Bacteria very rarely glycosylate proteins, and like other types of common hosts, such as yeasts, filamentous fungi, insect and plant cells, yield glycosylation patterns associated with rapid clearance from the blood stream, undesirable immune interactions, and in some specific cases, reduced biological activity. Among mammalian cells, Chinese hamster ovary (CHO) cells have been most commonly used during the last two decades. In addition to giving suitable glycosylation patterns, these cells allow consistent generation of genetically stable, highly productive clonal cell lines. They can be cultured to high densities in simple bioreactors using serum free media, and permit the development of safe and reproducible bioprocesses. Other commonly used animal cells include baby hamster kidney (BHK) cells, NSO- and SP2/0-mouse myeloma cells. More recently, production from transgenic animals has also been tested. (Jenkins, N., et al., Nature Biotechnol. 14 (1996) 975-981).

All antibodies contain carbohydrate structures at conserved positions in the heavy chain constant regions, with each isotype possessing a distinct array of N-linked carbohydrate structures, which variably affect protein assembly, secretion or functional activity (Wright, A., and Monison, S. L., Trends Biotechol. 15 (1997) 26-32). The structure of the attached N-linked carbohydrate varies considerably, depending on the degree of processing, and can include high-mannose, multiply-branched as well as biantennary complex oligosaccharides (Wright, A., and Morrison, S. L., Trends Biotechnol. 15 (1997) 26-32). Typically, there is heterogeneous processing of the core oligosaccharide structures attached at a particular glycosylation site such that even monoclonal antibodies exist as multiple glycoforms. Likewise, it has been shown that major differences in antibody glycosylation occur between cell lines, and even minor differences are seen for a given cell line grown under different culture conditions (Lifely, M. R., et al., Glycobiology 5(8) (1995) 813-822).

One way to obtain large increases in potency, while maintaining a simple production process and potentially avoiding significant, undesirable side effects, is to enhance the natural, cell-mediated effector functions of monoclonal antibodies by engineering their oligosaccharide component as described in Umana, P., et al., Nature Biotechnol. 17 (1999) 176-180 and U.S. Pat. No. 6,602,684. IgG1 type antibodies, the most commonly used antibodies in cancer immunotherapy, are glycoproteins that have a conserved N-linked glycosylation site at Asn297 in each CH2 domain. The two complex biantennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC) (Lifely, M. R., et al., Glycobiology 5(8) (1995) 813-822; Jefferis, R., et al., Immunol. Rev. 163 (1998) 59-76; Wright, A., and Morrison, S. L., Trends Biotechnol. 15 (1997) 26-32).

It was previously shown that overexpression in Chinese hamster ovary (CHO) cells of β(1,4)-N-acetylglucosaminyltransferase I11 (“GnTII17y), a glycosyltransferase catalyzing the formation of bisected oligosaccharides, significantly increases the in vitro ADCC activity of an antineuroblastoma chimeric monoclonal antibody (chCE7) produced by the engineered CHO cells (see Umana, P., et al., Nature Biotechnol. 17 (1999) 176-180; and WO1999/54342, the entire contents of which are hereby incorporated by reference). The antibody chCE7 belongs to a large class of unconjugated monoclonal antibodies which have high tumor affinity and specificity, but have too little potency to be clinically useful when produced in standard industrial cell lines lacking the GnTIII enzyme (Umana, P., et al., Nature Biotechnol. 17 (1999) 176-180). That study was the first to show that large increases of ADCC activity could be obtained by engineering the antibody producing cells to express GnTIII, which also led to an increase in the proportion of constant region (Fc)-associated, bisected oligosaccharides, including bisected, non-fucosylated oligosaccharides, above the levels found in naturally-occurring antibodies.

The term “cancer” as used herein includes lymphomas, lymphocytic leukemias, lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, including refractory versions of any of the above cancers, or a combination of one or more of the above cancers. In one embodiment, the term cancer refers to a CD20 expressing cancer.

The term “expression of the CD20” antigen is intended to indicate an significant level of expression of the CD20 antigen in a cell, preferably on the cell surface of a T- or B-cell, more preferably a B-cell, from a tumor or cancer, respectively, preferably a non-solid tumor. Patients having a “CD20 expressing cancer” can be determined by standard assays known in the art. For example, CD20 antigen expression can be measured using immunohistochemical (IHC) detection, FACS or via PCR-based detection of the corresponding mRNA.

The term “CD20 expressing cancer” as used herein refers to all cancers in which the cancer cells show an expression of the CD20 antigen. Preferably CD20 expressing cancer as used herein refers to lymphomas (preferably B-cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma) c) marginal zone lymphomas (including extranodal marginal zone B cell lymphoma (Mucosa-associated lymphatic tissue lymphomas, MALT), nodal marginal zone B cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma) f) hairy cell leukemia, g) lymphocytic lymphoma, waldenstrom's macroglobulinemia, h) acute lymphocytic leukemia (ALL), chronic lymphocytic leukemia (CLL)/small lymphocytic lymphoma (SLL), B-cell prolymphocytic leukemia, i) plasma cell neoplasms, plasma cell myeloma, multiple myeloma, plasmacytoma j) Hodgkin's disease.

In one embodiment, the CD20 expressing cancer is a B-Cell Non-Hodgkin's lymphomas (NHL). In another embodiment, the CD20 expressing cancer is a Mantle cell lymphoma (MCL), acute lymphocytic leukemia (ALL), chronic lymphocytic leukemia (CLL), B-cell diffuse large cell lymphoma (DLCL), Burkitt's lymphoma, hairy cell leukemia, follicular lymphoma, multiple myeloma, marginal zone lymphoma, post transplant lymphoproliferative disorder (PTLD), HIV associated lymphoma, waldenstrom's macroglobulinemia, or primary CNS lymphoma.

The term “a method of treating” or its equivalent, when applied to, for example, cancer refers to a procedure or course of action that is designed to reduce or eliminate the number of cancer cells in a patient, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of a patient, is nevertheless deemed to induce an overall beneficial course of action.

The terms “co-administration” or “co-administering” refer to the administration of said afucosylated anti-CD20, and fludarabine and/or mitoxantrone as one single formulation or as two separate formulations. The co-administration can be simultaneous or sequential in either order, wherein preferably there is a time period while both (or all) active agents simultaneously exert their biological activities. Said anti-CD20 afucosylated antibody and fludarabine and/or mitoxantrone are co-administered either simultaneously or sequentially (e.g. via an intravenous (i.v.) through a continuous infusion (one for the anti-CD20 antibody and eventually one for fludarabine and/or mitoxantrone. When both therapeutic agents are co-administered sequentially the dose is administered either on the same day in two separate administrations, or one of the agents is administered on day 1 and the second is co-administered on day 2 to day 7, preferably on day 2 to 4. Thus the term “sequentially” means within 7 days after the dose of the first component (fludarabine or mitoxantrone/or CD20 antibody), preferably within 4 days after the dose of the first component; and the term “simultaneously” means at the same time. The terms “co-administration” with respect to the maintenance doses of said afucosylated anti-CD20 antibody and fludarabine and/or mitoxantrone mean that the maintenance doses can be either co-administered simultaneously, if the treatment cycle is appropriate for both drugs, e.g. every week. Or fludarabine and/or mitoxantrone is e.g. administered e.g. every first to third day and said afucosylated antibody is administered every week. Or the maintenance doses are co-administered sequentially, either within one or within several days.

It is self-evident that the antibodies are administered to the patient in a “therapeutically effective amount” (or simply “effective amount”) which is the amount of the respective compound or combination that will elicit the biological or medical response of a tissue, system, animal or human that is being sought by the researcher, veterinarian, medical doctor or other clinician.

Depending on the type and severity of the disease, about 1 μg/kg to 50 mg/kg (e.g. 0.1-20 mg/kg) of said afucosylated anti-CD20 antibody and 1 μg/kg to 50 mg/kg (e.g. 0.1-20 mg/kg) of fludarabine and/or mitoxantrone is an initial candidate dosage for co-administration of both drugs to the patient In one embodiment the preferred dosage of said afucosylated anti-CD20 antibody (preferably the afucosylated humanized B-Ly1 antibody) will be in the range from about 0.05 mg/kg to about 30 mg/kg. Thus, one or more doses of about 0.5 mg/kg, 2.0 mg/kg, 4.0 mg/kg, 10 mg/kg or 30 mg/kg (or any combination thereof) may be co-administered to the patient. In one embodiment, the dosage of fludarabine and/or mitoxantrone will be in the range from 0.01 mg/kg to about 30 mg/kg, e.g. 0.1 mg/kg to 10.0 mg/kg. Depending on the type (species, gender, age, weight, etc.) and condition of the patient and on the type of afucosylated anti-CD20 antibody, the dosage and the administration schedule of said afucosylated antibody and fludarabine and/or mitoxantrone can differ. For example, the said afucosylated anti-CD20 antibody may be administered e.g. every one to three weeks and fludarabine and/or mitoxantrone may be administered daily or every 2 to 10 days. An initial higher loading dose, followed by one or more lower doses may also be administered.

In one embodiment, the dosage of said afucosylated anti-CD20 antibody (e.g., the afucosylated humanized B-Ly1 antibody) will be 400 to 1200 mg e.g., 400 to 800 mg) on day 1 of up to six 4-week-dosage-cycles and the dosage of fludarabine and/or mitoxantrone will be e.g. 20 mg/m² to 30 mg/m² (preferably 25 mg/m²) on day 1, 2 and 3 of up to six 4-week-dosage-cycles. Alternatively the preferred dosage of said afucosylated anti-CD20 antibody can be 400 to 1200 mg (preferably 400 to 800 mg) on day 1, 8, 15 of a 6-week-dosage-cycle and then in a dosage of 400 to 1200 mg (preferably 400 to 800 mg) on day 1 of up to five 4-week-dosage-cycles.

In one embodiment said humanized B-Ly1 antibody is administered in a dosage of 800 to 1600 mg on day 1 of up to six or seven 3- to 4-week-dosage-cycles, fludarabine is administered in a dosage of 20 mg/m² to 30 mg/m² (preferably 25 mg/m²) on day 1, 2 and 3 of up to six or seven 4-week-dosage-cycles (optionally plus an extra dose on Cycle 1 (day 8)).

In one embodiment, the afucosylated anti-CD20 antibody with an amount of fucose of 60% or less (preferably the afucosylated humanized B-Ly1 antibody) is used in the treatment of cancer in combination with fludarabine and cyclophosphamide (CTX; e.g. Cytoxan®). In one specific embodiment is the afucosylated anti-CD20 antibody with an amount of fucose is 60% or less (preferably the afucosylated humanized B-Ly1 antibody) for the manufacture of a medicament for the treatment of cancer in combination with fludarabine and cyclophosphamide (CTX; e.g. Cytoxan°). In such combination preferably said humanized B-Ly1 antibody is administered in a dosage of 800 to 1600 mg on day 1 of up to six or seven 3- to 4-week-dosage-cycles, fludarabine is administered in a dosage of 20 mg/m² to 30 mg/m² (preferably 25 mg/m²) on day 1, 2 and 3 of up to six or seven 4-week-dosage-cycles (optionally plus an extra dose on Cycle 1 (day 8)), and cyclophosphamide is administered in a dosage of 200 mg/m² to 300 mg/m² (preferably 250 mg/m²) on day 1, 2 and 3 of up to six or seven 4-week-dosage-cycles (optionally plus an extra dose on Cycle 1 (day 8)).

In one embodiment, the medicament is useful for preventing or reducing metastasis or further dissemination in such a patient suffering from cancer, preferably CD20 expressing cancer. The medicament is useful for increasing the duration of survival of such a patient, increasing the progression free survival of such a patient, increasing the duration of response, resulting in a statistically significant and clinically meaningful improvement of the treated patient as measured by the duration of survival, progression free survival, response rate or duration of response. In a preferred embodiment, the medicament is useful for increasing the response rate in a group of patients.

In the context of this invention, additional other cytotoxic, chemotherapeutic or anti-cancer agents, or compounds that enhance the effects of such agents (e.g. cytokines) may be used in the afucosylated anti-CD20 antibody and fludarabine and/or mitoxantrone combination treatment of cancer. Such molecules are suitably present in combination in amounts that are effective for the purpose intended. In one embodiment, the said afucosylated anti-CD20 antibody and fludarabine and/or mitoxantrone combination treatment is used without such additional cytotoxic, chemotherapeutic or anti-cancer agents, or compounds that enhance the effects of such agents.

Such agents include, for example: alkylating agents or agents with an alkylating action, such as cyclophosphamide (CTX; e.g. Cytoxan®), chlorambucil (CHL; e.g. Leukeran®), cisplatin (C is P; e.g. Platinol®) busulfan (e.g. Myleran®), melphalan, carmustine (BCNU), streptozotocin, triethylenemelamine (TEM), mitomycin C, and the like; anti-metabolites, such as methotrexate (MTX), etoposide (VP16; e.g. Vepesid®), 6-mercaptopurine (6 MP), 6-thiocguanine (6TG), cytarabine (Ara-C), 5-fluorouracil (5-FU), capecitabine (e.g. Xeloda®), dacarbazine (DTIC), and the like; antibiotics, such as actinomycin D, doxorubicin (DXR; e.g. Adriamycin®), daunorubicin (daunomycin), bleomycin, mithramycin and the like; alkaloids, such as vinca alkaloids such as vincristine (VCR), vinblastine, and the like; and other antitumor agents, such as paclitaxel (e.g. Taxol®) and paclitaxel derivatives, the cytostatic agents, glucocorticoids such as dexamethasone (DEX; e.g. Decadron®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. Ethyol®), dactinomycin, mechlorethamine (nitrogen mustard), streptozocin, cyclophosphamide, lomustine (CCNU), doxorubicin lipo (e.g. Doxil®), gemcitabine (e.g. Gemzar®), daunorubicin lipo (e.g. Daunoxome®), procarbazine, mitomycin, docetaxel (e.g. Taxotere®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil. Preferably the afucosylated anti-CD20 antibody and fludarabine and/or mitoxantrone combination treatment is used without such additional agents.

A preferred embodiment is the afucosylated anti-CD20 antibody with an amount of fucose is 60% or less (preferably the afucosylated humanized B-Ly1 antibody) for the treatment of cancer in combination with fludarabine and cyclophosphamide (CTX; e.g. Cytoxan®).

A preferred embodiment is the afucosylated anti-CD20 antibody with an amount of fucose is 60% or less (preferably the afucosylated humanized B-Ly1 antibody) for the manufacture of a medicament for the treatment of cancer in combination with fludarabine and cyclophosphamide (CTX; e.g. Cytoxan®).

The use of the cytotoxic and anticancer agents described above as well as antiproliferative target-specific anticancer drugs like protein kinase inhibitors in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

In the context of this invention, an effective amount of ionizing radiation may be carried out and/or a radiopharmaceutical may be used in addition to the afucosylated anti-CD20 antibody and fludarabine and/or mitoxantrone combination treatment of CD20 expressing cancer. The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111. Is also possible to label the antibody with such radioactive isotopes. Preferably the afucosylated anti-CD20 antibody and fludarabine and/or mitoxantrone combination treatment is used without such ionizing radiation.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anticancer agents. Parameters of adjuvant radiation therapies are, for example, contained in WO1999/60023.

The afucosylated anti-CD20 antibodies can be administered to a patient according to known methods, e.g., by intravenous administration as a bolus or by continuous infusion over a period of time, by intramuscular, intraperitoneal, intracerobrospinal, subcutaneous, intra-articular, intrasynovial, or intrathecal routes. In one embodiment, the administration of the antibody is intravenous or subcutaneous administration.

Fludarabine and/or mitoxantrone is administered to a patient according to known methods, e.g. by intravenous administration as a bolus or by continuous infusion over a period of time, by intramuscular, intraperitoneal, intracerobrospinal, subcutaneous, intra-articular, intrasynovial, intrathecal, or peroral routes. Intravenous or intraperitoneal administration is preferred.

As used herein, a “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

### Pharmaceutical Compositions:

Pharmaceutical compositions can be obtained by processing the anti-CD20 antibody and/or fludarabine and/or mitoxantrone according to this invention with pharmaceutically acceptable, inorganic or organic carriers. Lactose, corn starch or derivatives thereof, talc, stearic acids or it's salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

In embodiment, the composition comprisies both said afucosylated anti-CD20 antibody with an amount of fucose is 60% or less (preferably said afucosylated humanized B-Ly1 antibody) and fludarabine and/or mitoxantrone (preferably fludarabine) for use in the treatment of cancer, in particular of CD20 expressing cancer (e.g., a B-Cell Non-Hodgkin's lymphoma (NHL)).

Said pharmaceutical composition may further comprise one or more pharmaceutically acceptable carriers.

The present invention further provides a pharmaceutical composition, e.g., for use in cancer, comprising (i) an effective first amount of an afucosylated anti-CD20 antibody with an amount of fucose is 60% or less (preferably an afucosylated humanized B-Ly1 antibody), and (ii) an effective second amount of fludarabine and/or mitoxantrone. Such composition optionally comprises pharmaceutically acceptable carriers and/or excipients.

Pharmaceutical compositions of the afucosylated anti-CD20 antibody alone used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Pharmaceutical compositions of fludarabine and/or mitoxantrone (preferably fludarabine) can be similar to those described above for the afucosylated anti-CD20 antibody.

In one further embodiment of the invention, afucosylated anti-CD20 antibody and fludarabine and/or mitoxantrone are formulated into two separate pharmaceutical compositions.

The active ingredients may also be entrapped in microcapsules prepared, for example, by coacervation techniques or by interracial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

The present invention further provides a method for the treatment of cancer, comprising administering to a patient in need of such treatment (i) an effective first amount of an afucosylated anti-CD20 antibody with an amount of fucose is 60% or less, (preferably an afucosylated humanized B-Ly1 antibody); and (ii) an effective second amount of fludarabine and/or mitoxantrone.

In one embodiment, the amount of fucose of is between 40% and 60%.

Preferably said cancer is a CD20 expressing cancer.

Preferably said CD20 expressing cancer is a B-Cell Non-Hodgkin's lymphoma (NHL).

Preferably said afucosylated anti-CD20 antibody is a type II anti-CD20 antibody.

Preferably said antibody is a humanized B-Ly1 antibody.

Preferably said humanized B-Ly1 antibody is administered in a dosage of 400 to 1200 mg on day 1 of up to six 4-week-dosage-cycles, and fludarabine and/or mitoxantrone is administered in a dosage of 20 mg/m² to 30 mg/m² (preferably 25 mg/m²) on day 1, 2 and 3 of up to six 4-week-dosage-cycles.

In one embodiment, said method of treatment is characterized in that the treatment of cancer is in combination with fludarabine only (i.e. without mitoxantrone). In such combination preferably said humanized B-Ly1 antibody is administered in a dosage of 800 to 1600 mg on day 1 of up to six or seven 3- to 4-week-dosage-cycles, fludarabine is administered in a dosage of 20 mg/m² to 30 mg/m² (preferably 25 mg/m²) on day 1, 2 and 3 of up to six or seven 4-week-dosage-cycles (optionally plus an extra dose on Cycle 1 (day 8)), and cyclophosphamide is administered in a dosage of 200 mg/m² to 300 mg/m² (preferably 250 mg/m²) on day 1, 2 and 3 of up to six or seven 4-week-dosage-cycles (optionally plus an extra dose on Cycle 1 (day 8)).

In one embodiment, said method of treatment is characterized in that the treatment of cancer is in combination with fludarabine and cyclophosphamide (CTX; e.g. Cytoxan®) only (i.e. without mitoxantrone). In such combination preferably said humanized B-Ly1 antibody is administered in a dosage of 800 to 1600 mg on day 1 of up to six or seven 3- to 4-week-dosage-cycles, fludarabine is administered in a dosage of 20 mg/m² to 30 mg/m² (preferably 25 mg/m²) on day 1, 2 and 3 of up to six or seven 4-week-dosage-cycles (optionally plus an extra dose on Cycle 1 (day 8)), and cyclophosphamide is administered in a dosage of 200 mg/m² to 300 mg/m² (preferably 250 mg/m²) on day 1, 2 and 3 of up to six or seven 4-week-dosage-cycles (optionally plus an extra dose on Cycle 1 (day 8)).

In one embodiment, said method of treatment is characterized in that the treatment of cancer is in combination with mitoxantrone only.

As used herein, the term “patient” preferably refers to a human in need of treatment with an afucosylated anti-CD20 antibody (e.g. a patient suffering from CD20 expressing cancer) for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others.

The invention further comprises an afucosylated anti-CD20 antibody with an amount of fucose is 60% or less, and fludarabine and/or mitoxantrone (preferably fludarabine) for use in the treatment of cancer. In such combination preferably said humanized B-Ly1 antibody is administered in a dosage of 800 to 1600 mg on day 1 of up to six or seven 3- to 4-week-dosage-cycles, fludarabine is administered in a dosage of 20 mg/m² to 30 mg/m² (preferably 25 mg/m²) on day 1, 2 and 3 of up to six or seven 4-week-dosage-cycles (optionally plus an extra dose on (day 8)), and cyclophosphamide is administered in a dosage of 200 mg/m² to 300 mg/m² (preferably 250 mg/m²) on day 1, 2 and 3 of up to six or seven 4-week-dosage-cycles (optionally plus an extra dose on Cycle 1 (day 8)). Preferably said combination is without mitoxantrone.

In one embodiment, the afucosylated anti-CD20 antibody with an amount of fucose is 60% or less (preferably the afucosylated humanized B-Ly1 antibody) is used in the treatment of cancer in combination with fludarabine and cyclophosphamide (CTX; e.g. Cytoxan®). In such combination preferably said humanized B-Ly1 antibody is administered in a dosage of 800 to 1600 mg on day 1 of up to six or seven 3- to 4-week-dosage-cycles, fludarabine is administered in a dosage of 20 mg/m² to 30 mg/m² (preferably 25 mg/m²) on day 1, 2 and 3 of up to six or seven 4-week-dosage-cycles (optionally plus an extra dose on Cycle 1 (day 8)), and cyclophosphamide is administered in a dosage of 200 mg/m² to 300 mg/m² (preferably 250 mg/m²) on day 1, 2 and 3 of up to six or seven 4-week-dosage-cycles (optionally plus an extra dose on Cycle 1 (day 8)).

Preferably said afucosylated anti-CD20 antibody is a humanized B-Ly1 antibody.

Preferably the cancer is a CD20 expressing cancer, more preferably a B-Cell Non-Hodgkin's lymphoma (NHL).

The patents, patent applications and other published documents cited herein are incorporated by reference in their entirety.

The following examples, sequence listing, and figures are provided to aid the understanding of the present invention, the true scope of which is set forth in the appended claims. It is understood that modifications can be made in the procedures set forth without departing from the spirit of the invention.

### Sequence Listing

SEQ ID NO: 1 amino acid sequence of variable region of the heavy chain (VH) of murine monoclonal anti-CD20 antibody B-Ly1 .
SEQ ID NO: 2 amino acid sequence of variable region of the light chain (VL) of murine monoclonal anti-CD20 antibody B-Ly1 .
SEQ ID NO: 3-19 amino acid sequences of variable region of the heavy chain (VH) of humanized B-Ly1 antibodies (B-HH2 to B-HH9, B-HL8, and B-HL10 to B-HL17)
SEQ ID NO: 20 amino acid sequences of variable region of the light chain (VL) of humanized B-Ly1 antibody B-KV1

### DESCRIPTION OF THE FIGURES

FIG. 1 In vivo antitumor activity of combined treatment of an afucosylated type II anti-CD20 antibody (B-HH6-B-KV1 GE) with fludarabine (in comparison with combination of rituximab (=focusylated type I anti-CD20 antibody) with fludarabine and in comparison with the respective monotherapies.

FIG. 2 Combination of an afucosylated type II anti-CD20 antibody (B-HH6-B-KV1 GE=humanized B-Ly1, glycoengineered) (1 μg/ml) and Fludarabine (0.25 μg/ml) from Oh to 72 h in Rec1 MCL cells. Values for the untreated control were normed to 100%.

FIG. 3 Combination of an afucosylated type II anti-CD20 antibody (B-HH6-B-KV1 GE=humanized B-Ly1, glycoengineered) (1 μg/ml) and Fludarabine (0.25 μg/ml) from 0 h to 72 h in Z138 MCL cells. Values for the untreated control were normed to 100%.

FIG. 4 Combination of an afucosylated type II anti-CD20 antibody (B-HH6-B-KV1 GE=humanized B-Ly1, glycoengineered) (1 μg/ml) and Mitoxantrone (0.5 μg/ml) from 0 h to 72 h in Granta-519 MCL cells. Values for the untreated control were normed to 100%.

FIG. 5 Combination of an afucosylated type II anti-CD20 antibody (B-HH6-B-KV1 GE=humanized B-Ly1, glycoengineered) (1 μg/ml) and Mitoxantrone (0.25 μg/ml) from Oh to 72 h in Rec-1 MCL cells. Values for the untreated control were normed to 100%.

### EXPERIMENTAL PROCEDURES

### Example 1

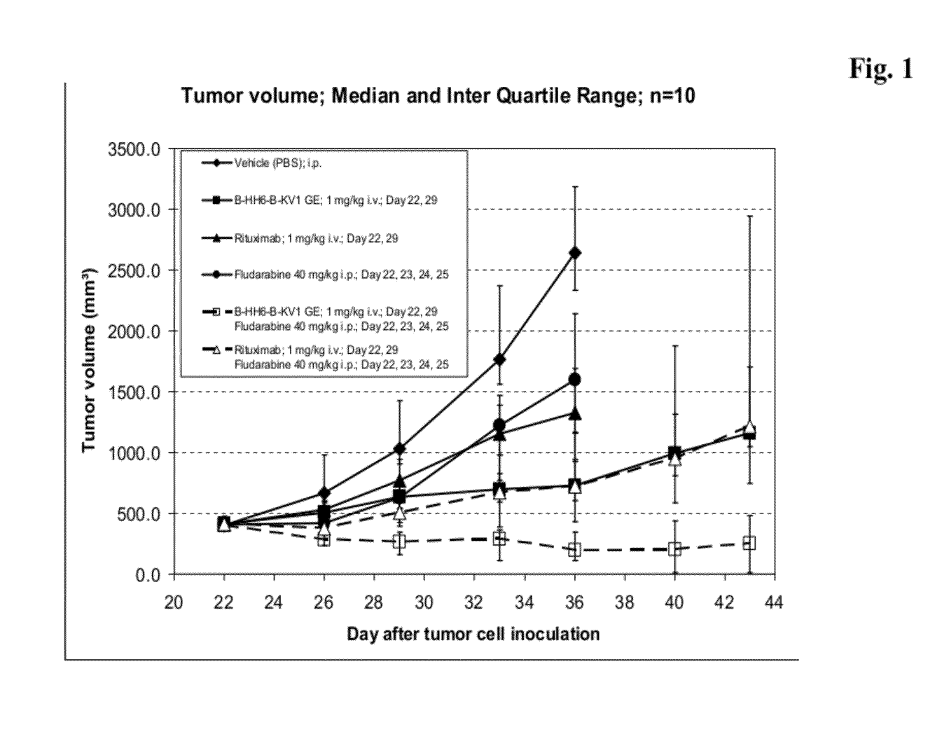

### See FIG. 1

In Vivo Antitumor Activity of Combined Treatment of an Afucosylated Type II Anti-CD20 Antibody (B-HH6-B-KV1 GE) with Fludarabine

### Test Agents

Type II anti-CD20 antibody B-HH6-B-KV1 GE (=humanized B-Ly1, glycoengineered B-HH6-B-KV1, see WO2005/044859 and WO2007/031875) was provided as stock solution (c=9.4 mg/ml) from GlycArt, Schlieren, Switzerland. Antibody buffer included histidine, trehalose and polysorbate 20. Antibody solution was diluted appropriately in PBS from stock for prior injections.

Fludarabinephosphate (Fludarabinmedac) was purchased from medac, Gesellschaft für klinische Spezialpräparate mbH, Fehlandstr. 3, 20354 Hamburg, Germany. Required dilutions were adjusted from the manufactured stock solution of 25 mg/ml.

### Cell Lines and Culture Conditions

The human Z138 mantle cell lymphoma cell line was routinely cultured in DMEM supplemented with 10% fetal bovine serum (PAA Laboratories, Austria) and 2 mM L-glutamine at 37° C. in a water-saturated atmosphere at 8% CO2. Passage 2 was used for transplantation. Cells were co-injected with Matrigel.

### Animals

Female SCID beige mice; age 4-5 weeks at arrival (purchased from Charles River, Sulzfeld, Germany) were maintained under specific-pathogen-free condition with daily cycles of 12 h light/12 h darkness according to committed guidelines (GV-olas; Felasa; TierschG). Experimental study protocol was reviewed and approved by local government. After arrival animals were maintained in the quarantine part of the animal facility for two weeks to get accustomed to new environment and for observation. Continuous health monitoring was carried out on regular basis. Diet food (Provimi Kliba 3337) and water (acidified pH 2.5-3) were provided ad libitum.

### Monitoring

Animals were controlled daily for clinical symptoms and detection of adverse effects. For monitoring throughout the experiment body weight of animals was documented two times weekly and tumor volume was measured by caliper after staging.

### Treatment of Animals

Animal treatment started at the day of randomisation 22 days after tumor cell inoculation. Humanized type II anti-CD20 antibody B-HH6-B-KV1 GE or Rituximab were administered as single agents i.v. q7d on study day 22 and 29 at the indicated dosage of 1 mg/kg. The corresponding vehicle was administered on the same days. Fludarabine was given i.p. on day 22, 23, 24, and 25 at 40 mg/kg. In the combination therapy groups, the chemotherapeutic agent was administered 8 hours after both antibodies on day 22.

### Tumor Growth Inhibition Study In Vivo (See FIG. 1)

On day 36 after tumor cell inoculation, there was a tumor growth inhibition of 50%, 60%, 85%, 86% or 108% (regression) in the animals given fludarabine, rituximab, combination of rituximab and fludarabine, anti-CD20 antibody B-HH6-B-KV1 GE or combination of anti-CD20 antibody and fludarabine, respectively, compared to the control group. In the treatment group with combination of anti-CD20 antibody and fludarabine even 3 out of 10 animals were tumor free or showed a nearly complete loss of tumor burden (n=2) on day 43.

### Conclusions:

These in vivo results demonstrate that the afucosylated type II anti-CD20 antibody B-HH6-B-KV1 GE (=humanized B-Ly1, glycoengineered) in combination with the chemotherapeutical compound Fludarabine shows more than additive activity on NHL Z138 xenograft.

### Example 2

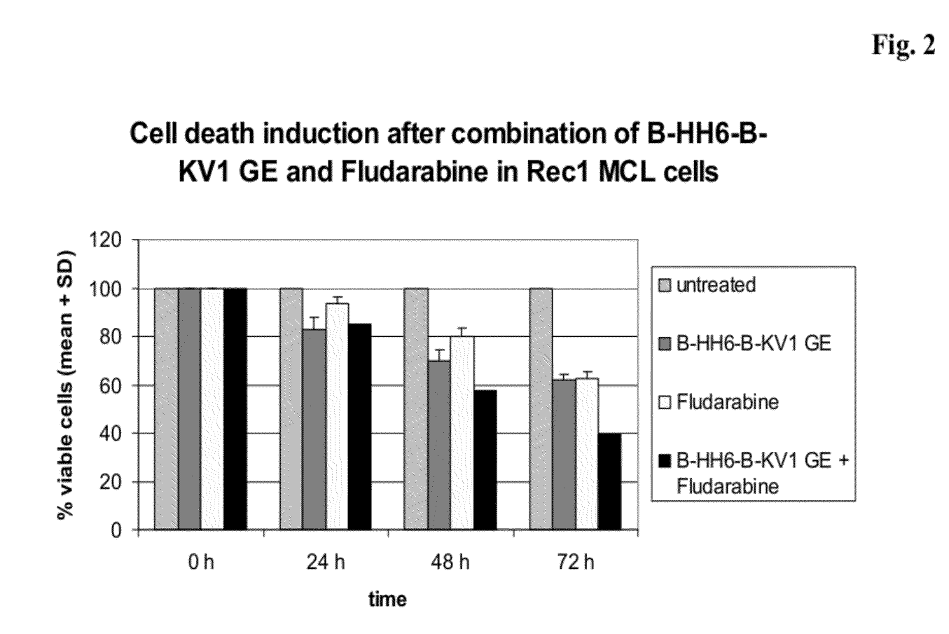

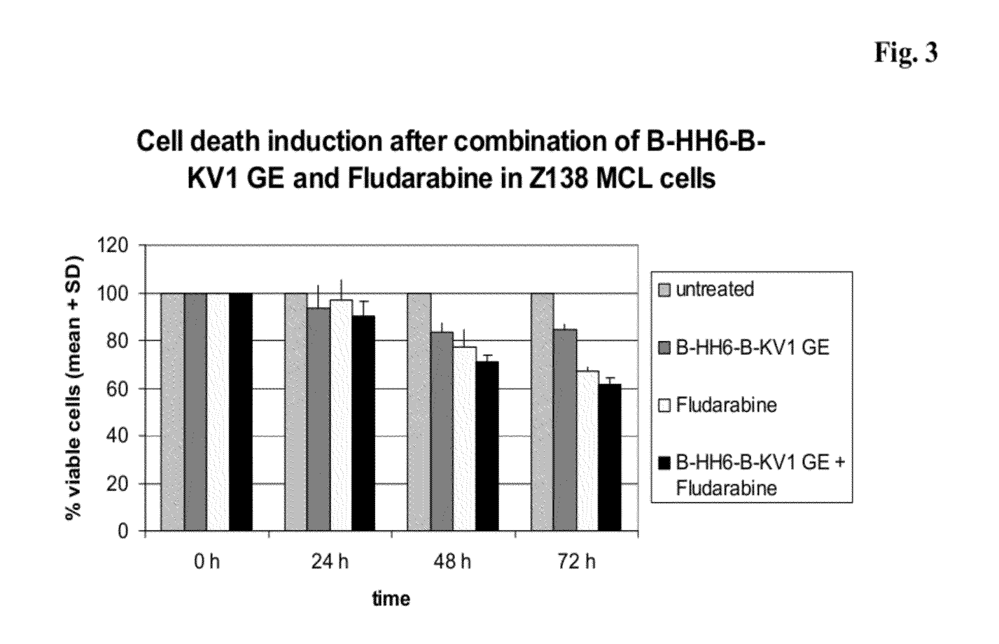

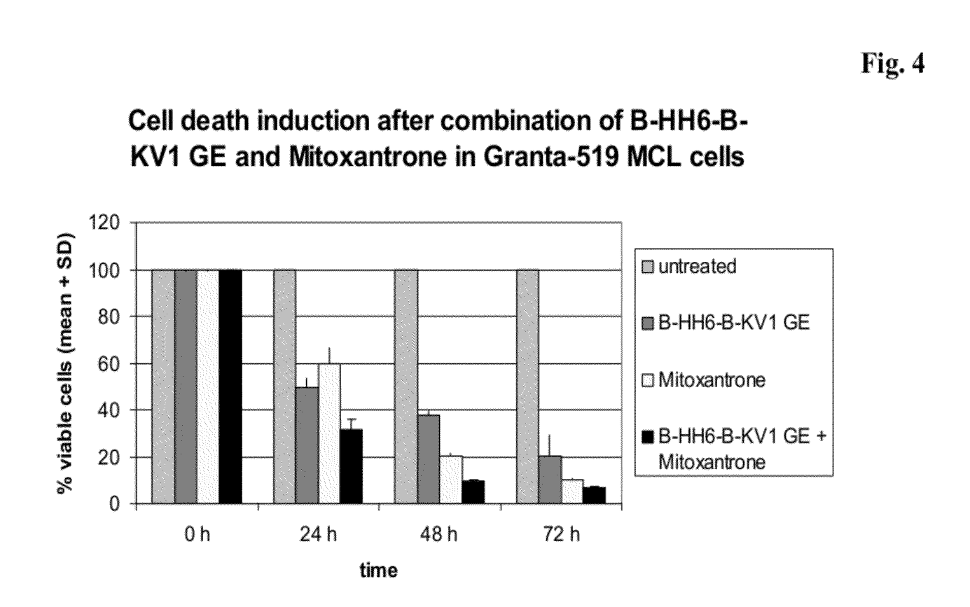

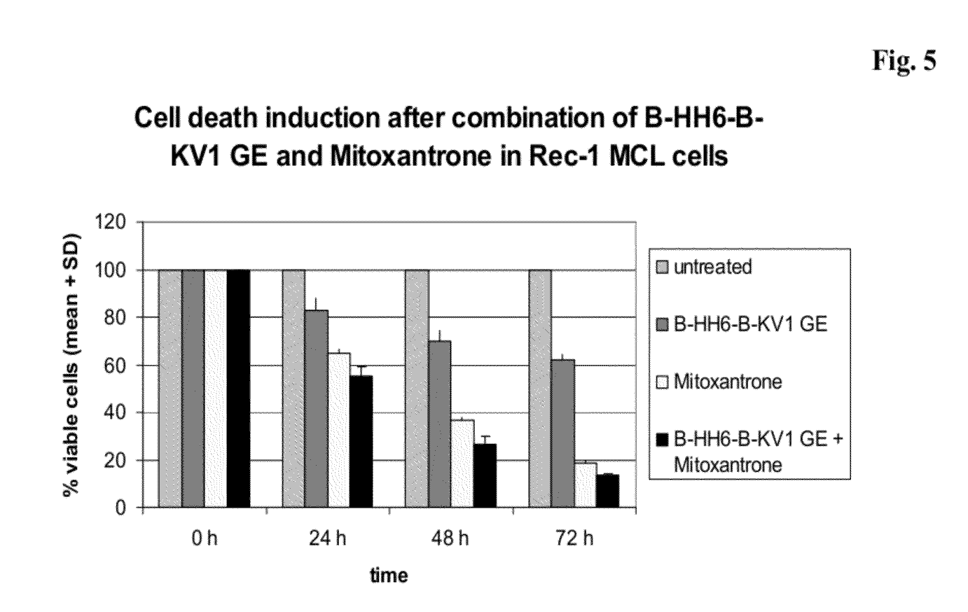

### See FIGS. 2 to 5

In Vitro Evaluation of the Antiproliferative Activity of an Afucosylated Type II Anti-CD20 Antibody (B-HH6-B-KV1 GE) with Fludarabine or Mitoxantrone

### Material and Methods

### Characterization of the Applied Tumor Cell Lines

The following Non-Hodgkin Lymphoma (NHL) cell lines were used in the experiments: Granta-519, HBL-2, Rec-1 and Z-138 as Mantle Cell Lymphoma cell lines and a Karpas-422 as a Diffuse Large B-Cell Lymphoma cell line. All cell lines were obtained from the “Deutsche Sammlung von Mikroorganismen and Zellkulturen GmbH” (DSMZ), Braunschweig, Germany.

### Cell Culture Conditions:

All cell lines were stored according to standard procedures and cultivated at 37° C., 5% CO2- and 95% relative humidity in a CO2 incubator. Granta 519, HBL-2, JeKo-1, Rec-1 and Z-138 were cultivated in RPMI-1640 medium with 10% heat-inactivated FCS and 1% Penicillin/Streptomycin.

### Determination of Viability and Proliferation Using the Trypan Blue Cell Exclusion Method

The density of viable cells was determined with BeckmanCoulter ViCell™ Cell Viability Analyzer on the basis of the trypan blue cell exclusion method. The test is based on the principle that viable cells have an intact cell membrane that blocks the uptake of trypan blue whereas dead cells have lost this ability. Therefore viable cells have a transparent cytoplasm whereas dead cells can be identified due to their blue cytoplasm.

### Materials:

| | |
|---|---|
| Fetal calf serum | 0.1 μm sterile filtered, origin: South America, heat |
| | inactivated at 56° C.; (PAN ™ Biotech GmbH) |
| Penicillin/ | 10.000 U Pen/ml; 10 mg Streptomycin/ml; (PAN ™ |
| Streptomycin | Biotech GmbH) |
| Pipette Tips | 1-200 μl; 101-1000 μl: Natural Graduated Pipette Tips, |
| | RNAse, DNAse, DNA and Pyrogen free; (Starlab |
| | GmbH) |
| RPMI-1640 | L-Glutamine with 2.0 g/l NaHCO₃, sterile filtered, |
| | Vol. 500 ml; (PAN ™ Biotech GmbH) |
| Tissue Flasks | PE Vented Cap Green; 25, 75, 150 cm³; (Sarstedt, |
| | Inc.) |
| Vi-Cell ™ AS | Cell Viability Analyzer; BeckmanCoulter |
| Vi-Cell ™ | Biodegradable, azide-free reagent, contains a |
| Cleaning | proteolytic enzyme, |
| Agent | |
| Vi-Cell ™ Buffer | Part # 383202 |
| Solution | |
| Vi-Cell ™ | Part # 383200; 0.4% in NaCl |
| Trypan Blue | |
| Vi-Cell ™ | Part # 383201 |
| Disinfectant | |

### Test Compounds:

B-HH6-B-KV1 GE (=humanized B-Ly1, glycoengineered B-HH6-B-KV1, see WO2005/044859 and WO2007/031875):
   Stock solution 10 mg/ml (Roche, Glycart)
Combinations partners
   1. Fludarabine: Stock solution 25 mg/ml in PBS; Medac GmbH (Wedel)
   2. Mitoxantrone: Stock solution 2 mg/ml (Baxter Oncology GmbH)

### Experimental Protocol:

Using a MCL cell line panel (Granta-519, HBL-2, Jeko-1, Rec-1 and Z-138) and a Diffuse Large B-Cell Lymphoma cell line (Karpas-422) the effect of B-HH6-B-KV1 GE alone as well as in combination with Fludarabine, Mitoxantrone on cell proliferation and viability was determined. Trypan-blue exclusion tests were used to analyze cell viability.

Briefly, MCL cells were diluted to a start density of 0.5×10⁶ cells/ml in 6 ml total volume corresponding to a total cell number of 3×10⁶ cells and treated with 1 μg/ml B-HH6-B-KV1 GE in combination with the subsequent concentrations of the chemotherapeutics Fludarabine or Mitoxantrone. These concentrations were determined in pre-experiments on MCL cells
1. 0.25 μg/ml Fludarabine
2. 0.25 and 0.5 μg/ml Mitoxantrone

1 ml samples were taken at 0 h, 24 h, 48 h, 72 h every day and the number of viable cells was determined. The reduction of cell proliferation was used for a fractional product calculation (synergism >0.1; additive effect −0.1<x<0.1; antagonism <−0.1). Experiments were independently performed three times.

### Results:

After mono-exposure with B-HH6-B-KV1 GE (1 μg/ml), Granta-519 and Rec-1 showed the highest sensitivity (Granta: 65-75% cell reduction, Rec-1: 30-45%). Intermediate results were achieved for HBL-2 (20-30%), Z-138 and Karpas-422 (10-15%), Jeko-1 (5%). Fludarabine alone resulted in a 20-40% cell reduction, whereas Mitoxantrone treatment demonstrated a high impact on all cell lines (80-95% cell reduction).

The combination of B-HH6-B-KV1 GE with the respective agents showed additive effects for all combinations resulting in 40-80% cell reduction (Fludarabine) and 85-95% (Mitoxantrone).

### Conclusions:

These in vitro results demonstrate that the afucosylated type II anti-CD20 antibody B-HH6-B-KV1 GE (=humanized B-Ly1, glycoengineered) in combination with the chemotherapeutical compounds Fludarabine and Mitoxantrone shows promising activity on NHL cell lines (e.g. more than additive in some cases).

It is claimed:

1. Use of an afucosylated anti-CD20 antibody with an amount of fucose of 60% or less of the total amount of oligosaccharides (sugars) at Asn297, for the manufacture of a medicament for the treatment of cancer in combination with fludarabine and/or mitoxantrone.

2. Use according to any one of claims 1 to 2 characterized in that said cancer is a B-Cell Non-Hodgkin's lymphoma (NHL).

3. Use according to claim 1, characterized in that said antibody is a humanized B-Ly1 antibody.

4. Use according to claim 3, characterized in that the treatment of cancer is in combination with fludarabine.

5. Use according to claim 4 characterized in that the humanized B-Ly1 antibody is administered in a dosage of 800 to 1600 mg on day 1 of up to six or seven 3- to 4-week-dosage-cycles, and fludarabine is administered in a dosage of 20 mg/m² to 30 mg/m² on day 1, 2 and 3 of up to six or seven 4-week-dosage-cycles.

6. Use according to claim 3, characterized in that the treatment of cancer is in combination with fludarabine and cyclophosphamide.

7. Use according to any one of claim 6 characterized in that the humanized B-Ly1 antibody is administered in a dosage of 800 to 1600 mg on day 1 of up to six or seven 3- to 4-week-dosage-cycles, fludarabine is administered in a dosage of 20 mg/m² to 30 mg/m² on day 1, 2 and 3 of up to six or seven 4-week-dosage-cycles, and cyclophosphamide is administered in a dosage of 200 mg/m² to 300 mg/m² on day 1, 2 and 3 of up to six or seven 4-week-dosage-cycles.

8. Use according to any one of claims 1 to 3, characterized in that the treatment of cancer is in combination with mitoxantrone.

9. Use according to any one of claims 1 to 8, characterized in that one or more additional other cytotoxic, chemotherapeutic or anti-cancer agents, or compounds or ionizing radiation that enhance the effects of such agents are administered.

10. A composition comprising an afucosylated anti-CD20 antibody with an amount of fucose of 60% or less of the total amount of oligosaccharides (sugars) at Asn297 and fludarabine and/or mitoxantrone for the treatment of cancer.

11. A method of treatment of patient suffering from cancer by administering an afucosylated anti-CD20 antibody with an amount of fucose of 60% or less of the total amount of oligosaccharides (sugars) at Asn297 in combination with fludarabine and/or mitoxantrone, to a patient in the need of such treatment.

12. The method of treatment according to claim 10, characterized in that said antibody is a humanized B-Ly1 antibody.

13. The method of treatment according to claim 11, characterized in that the treatment of cancer is in combination with fludarabine and cyclophosphamide.

14. The method of treatment according to claim 12, characterized in that the humanized B-Ly1 antibody is administered in a dosage of 800 to 1600 mg on day 1 of up to six or seven 3- to 4-week-dosage-cycles, fludarabine is administered in a dosage of 20 mg/m² to 30 mg/m² on day 1, 2 and 3 of up to six or seven 4-week-dosage-cycles, and cyclophosphamide is administered in a dosage of 200 mg/m² to 300 mg/m² on day 1, 2 and 3 of up to six or seven 4-week-dosage-cycles.


SEQUENCE LISTING (20 sequences)

SEQ ID NO: 1 - protein, 112 aa, Mus sp.
  MISC_FEATURE: amino acid sequence of variable region of the heavy chain (VH) of murine monoclonal anti-CD20 antibody B-Ly1
GPELVKPGASVKISCKASGYAFSYSWMNWVKLRPGQGLEWIGRIFPGDGDTDYNGKFKGKATLTADKSSNTAYMQLTSLTSVDSAVYLCARNVFDGYWLVYWGQGTLVTVSA

SEQ ID NO: 2 - protein, 103 aa, Mus sp.
  MISC_FEATURE: amino acid sequence of variable region of the light chain (VL) of murine monoclonal anti-CD20 antibody B-Ly1
NPVTLGTSASISCRSSKSLLHSNGITYLYWYLQKPGQSPQLLIYQMSNLVSGVPDRFSSSGSGTDFTLRISRVEAEDVGVYYCAQNLELPYTFGGGTKLEIKR

SEQ ID NO: 3 - protein, 119 aa, Artificial
  amino acid sequences of variable region of the heavy chain (VH) of humanized B-Ly1 antibody (B-HH2)
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 4 - protein, 119 aa, Artificial
  amino acid sequences of variable region of the heavy chain (VH) of humanized B-Ly1 antibody (B-HH3)
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYLCARNVFDGYWLVYWGQGTLVTVSS

SEQ ID NO: 5 - protein, 119 aa, Artificial
  amino acid sequences of variable region of the heavy chain (VH) of humanized B-Ly1 antibody (B-HH4)
QVQLVQSGAEVKKPGASVKVSCKVSGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 6 - protein, 119 aa, Artificial
  amino acid sequences of variable region of the heavy chain (VH) of humanized B-Ly1 antibody (B-HH5)
QVQLVQSGAEVKKPGSSVKVSCKASGYAFSYSWMSWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 7 - protein, 119 aa, Artificial
  amino acid sequences of variable region of the heavy chain (VH) of humanized B-Ly1 antibody (B-HH6)
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 8 - protein, 119 aa, Artificial
  amino acid sequences of variable region of the heavy chain (VH) of humanized B-Ly1 antibody (B-HH7)
QVQLVQSGAEVKKPGSSVKVSCKASGYAFSYSWISWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 9 - protein, 119 aa, Artificial
  amino acid sequences of variable region of the heavy chain (VH) of humanized B-Ly1 antibody (B-HH8)
QVQLVQSGAEVKKPGASVKVSCKASGYTFTYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 10 - protein, 119 aa, Artificial
  amino acid sequences of variable region of the heavy chain (VH) of humanized B-Ly1 antibody (B-HH9)
QVQLVQSGAEVKKPGASVKVSCKASGYTFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 11 - protein, 119 aa, Artificial
  amino acid sequences of variable region of the heavy chain (VH) of humanized B-Ly1 antibody (B-HL8)
EVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 12 - protein, 119 aa, Artificial
  amino acid sequences of variable region of the heavy chain (VH) of humanized B-Ly1 antibody (B-HL10)
EVQLVESGGGLVKPGGSLRLSCAASGFAFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 13 - protein, 119 aa, Artificial
  amino acid sequences of variable region of the heavy chain (VH) of humanized B-Ly1 antibody (B-HL11)
QVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 14 - protein, 119 aa, Artificial
  amino acid sequences of variable region of the heavy chain (VH) of humanized B-Ly1 antibody (B-HL12)
EVQLVESGAGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 15 - protein, 119 aa, Artificial
  amino acid sequences of variable region of the heavy chain (VH) of humanized B-Ly1 antibody (B-HL13)
EVQLVESGGGVVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 16 - protein, 119 aa, Artificial
  amino acid sequences of variable region of the heavy chain (VH) of humanized B-Ly1 antibody (B-HL14)
EVQLVESGGGLKKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 17 - protein, 119 aa, Artificial
  amino acid sequences of variable region of the heavy chain (VH) of humanized B-Ly1 antibody (B-HL15)
EVQLVESGGGLVKPGSSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 18 - protein, 119 aa, Artificial
  amino acid sequences of variable region of the heavy chain (VH) of humanized B-Ly1 antibody (B-HL16)
EVQLVESGGGLVKPGGSLRVSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 19 - protein, 119 aa, Artificial
  amino acid sequences of variable region of the heavy chain (VH) of humanized B-Ly1 antibody (B-HL17)
EVQLVESGGGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 20 - protein, 115 aa, Artificial
  amino acid sequences of variable region of the light chain (VL) of humanized B-Ly1 antibody B-KV1
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV